(19) United States
(12) Patent Application Publication
(10) Pub. No.: US 2007/0226815 A1
(43) Pub. Date: Sep. 27, 2007

(54) Interferon-alpha induced gene

(75) Inventors: Jean-Francois Meritet, Paris (FR); Michel Dron, Bourg la Reine (FR); Michael Gerard Tovey, Paris (FR)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Pharma Pacific Pty Ltd., Laverton North (AU)

(21) Appl. No.: 11/588,592

(22) Filed: Oct. 27, 2006

Related U.S. Application Data
(63) Continuation of application No. 10/550,768, now abandoned.

(30) Foreign Application Priority Data
Mar. 27, 2003 (GB) 0307127.1

(51) Int. Cl. A01K 67/027; C12Q 1/68; C07H 21/04; C12P 21/04; A61K 38/21; C07K 14/56

(52) U.S. Cl. 800/14; 435/6; 424/85.4; 435/320.1; 435/325; 435/69.51; 530/351; 536/23.5

(57) ABSTRACT

The present invention relates to identification of a gene upregulated by interferon-a administration corresponding to the cDNA sequence set forth in SEQ. ID. NO. 1. Determination of expression products of this gene is proposed as having utility in predicting responsiveness to treatment with interferon-α and other interferons which act at the Type 1 interferon receptor. Therapeutic use of the protein encoded by the same geneis also envisaged.

### FIELD OF THE INVENTION

The present invention relates to identification of a human gene upregulated by interferon-α (IFN-α) administration, the coding sequence of which is believed to be previously unknown. Detection of expression products of this gene may find use in predicting responsiveness to IFN-α and other interferons which act at the Type 1 interferon receptor. Therapeutic use of the isolated novel protein encoded by the same gene is also envisaged.

### BACKGROUND OF THE INVENTION

IFN-α is widely used for the treatment of a number of disorders. Disorders which may be treated using IFN-α include neoplastic diseases such as leukemia, lymphomas, and solid tumours, AIDS-related Kaposi's sarcoma and viral infections such as chronic hepatitis. IFN-α has also been proposed for admin ion via the oromucosal route for the treatment of autoimmune, mycobacterial, neurodegenerative, parasitic and viral disease. In particular, IFN-α has been proposed, for example, for the treatment of multiple sclerosis, leprosy, tuberculosis, encephalitis, malaria, cervical cancer, genital herpes, hepatitis B and C, HIV, HPV and HSV-1 and 2. It has also been suggested for the treatment of arthritis, lupus and diabetes. Neoplastic diseases such as multiple myeloma, hairy cell leukemia, chronic myelogenous leukemia, low grade lymphoma, cutaneous T-cell lymphoma, carcinoid tumours, cervical cancer, sarcomas including Kaposi's sarcoma, kidney tumours, carcinomas including renal cell carcinoma, hepatic cellular carcinoma, nasopharyngeal carcinoma, haematological malignancies, colorectal cancer, glioblastoma, laryngeal papillomas, lung cancer, colon cancer, malignant melanoma and brain tumours are also suggested as being treatable by administration of IFN-α via the oromucosal route, i.e. the oral route or the nasal route.

IFN-α is a member of the Type 1 interferon family, which exert their characteristic biological activities through interaction with the Type 1 interferon receptor. Other Type 1 interferons include IFN-β, IFN-ω and IFN-τ.

Unfortunately, not all potential patients for treatment with a Type 1 interferon such as interferon-α, particularly, for example, patients suffering from chronic viral hepatitis, neoplastic disease and relapsing remitting multiple sclerosis, respond favourably to Type 1 interferon therapy and only a fraction of those who do respond exhibit long-term benefit. The inability of the physician to confidently predict the therapeutic outcome of Type 1 interferon treatment raises serious concerns as to the cost-benefit ratio of such treatment, not only in terms of wastage of an expensive biopharmaceutical and lost time in therapy, but also in terms of the serious side effects to which the patient is exposed. Furthermore, abnormal production of IFN-α has been shown to be associated with a number of autoimmune diseases. For these reasons, there is much interest in identifying Type 1 interferon responsive genes since Type 1 interferons exert their therapeutic action by modulating the expression of a number of genes. Indeed, it is the specific pattern of gene expression induced by Type 1 interferon treatment that determines whether a patient will respond favourably or not to the treatment

### SUMMARY OF THE INVENTION

A human gene cDNA has now been identified as corresponding to a mouse gene upregulated by administration of IFN-α by an oromucosal route or intraperitoneally and is believed to represent a novel DNA. The corresponding human gene is thus now also designated an IFN-α upregulated gene.

The protein encoded by the same gene has a molecular weight of 198 kDa and is referred to below as HuIFRG 198 protein This protein, and functional variants thereof, are now envisaged as therapeutic agents, in particular for use as an anti-viral, anti-tumour or immunomodulatory agent. For example, they may be used in the treatment of autoimmune, mycobacterial, neurodegenerative, parasitic or viral disease, arthritis, diabetes, lupus, multiple sclerosis, leprosy, tuberculosis, encephalitis, malaria, cervical cancer, genital herpes, hepatitis B or C, HIV, HPV, HSV-1 or 2, or neoplastic disease such as multiple myeloma, hairy cell leukemia, chronic myelogenous leukemia, low grade lymphoma, cutaneous T-cell lymphoma, carcinoid tumours, cervical cancer, sarcomas including Kaposi's sarcoma, kidney tumours, carcinomas including renal cell carcinoma, hepatic cellular carcinoma, nasopharyngeal carcinoma, haematological malignancies, colorectal cancer, glioblastoma, laryngeal papillomas, lung cancer, colon cancer, malignant melanoma or brain tumours. In other words, such a protein may find use in treating any Type 1 interferon treatable disease.

Determination of the level of HuIFRG 198 protein or a naturally-occurring variant thereof, or the corresponding mRNA, in cell samples of Type 1 interferon-treated patients, e.g. patients treated with IFN-α, e.g. such as by the oromucosal route or intravenously, may also be used to predict responsiveness to such treatment. It has additionally been found that alternatively, and more preferably, such responsiveness may be judged, for example, by treating a sample of human peripheral blood mononuclear cells in vitro with a Type 1 interferon and looking for upregulation or downregulation of an expression product, preferably mRNA, corresponding to the HuIFRG 198 gene.

According to a first aspect of the invention, there is thus provided an isolated polypeptide comprising;
(i) the amino acid sequence of SEQ ID NO: 2;
(ii) a variant thereof having substantially similar function, e.g. an immunomodulatory activity and/or an anti-viral activity and/or an anti-tumour activity; or
(iii) a fragment of (i) or (ii) which retains substantially similar function, e.g. an immunomodulatory activity and/or an anti-viral activity and/or an anti-tumour activity.

In general, proteins of most interest are those having greater than 98% identity with the amino acid sequence of SEQ ID NO: 2 over the full length of SEQ ID NO: 2.

The invention also provides such a protein for use in therapeutic treatment of a human or non-human animal, more particularly for use as an anti-viral, anti-tumour or immunomodulatory agent. As indicated above, such use may extend to any Type 1 interferon treatable disease.

According to another aspect of the invention, there is provided an isolated polynucleotide encoding a polypeptide of the invention as defined above or a complement thereof. Such a polynucleotide will typically include a sequence comprising:
(a) the nucleic acid of SEQ. ID. No. 1 or the coding sequence thereof and/or a s sequence complementary thereto;
(b) a sequence which hybridises, e.g. under stringent conditions, to a sequence complementary to a sequence as defined in (a);
(c) a sequence which is degenerate as a result of the genetic code to a sequence as defined in (a) or (b);
(d) a sequence having at least 60% identity to a sequence as defined in (a), (b) or (c).

Preferred polynucleotides are those which encode a polypeptide having more than 98% identity with the sequence of SEQ ID NO: 2 over the full length of SEQ ID NO: 2.

The invention also provides;

an expression vector which comprises a polynucleotide of the invention and which is capable of expressing a polypeptide of the invention;
a host cell containing an expression vector of the invention;
an antibody specific for a polypeptide of the invention;
a method of treating a subject having a Type 1 interferon treatable disease, which method comprises administering to the said patient an effective amount of HuIFRG 198 protein or a functional variant thereof
use of such a polypeptide in the manufacture of a medicament for use in therapy as an anti-viral or anti-tumour or immunomodulatory agent, more particularly for use in treatment of a Type 1 interferon treatable disease;
a pharmaceutical composition comprising a polypeptide of the invention and a pharmaceutically acceptable carrier or diluent;
a method of producing a polypeptide of the invention, which method comprises maintaining host cells of the invention under conditions suitable for obtaining expression of the polypeptide and isolating the said polypeptide;
a polynucleotide of the invention, e.g. in the form of an expression vector, which directs expression in vivo of a polypeptide as defined above for use in therapeutic treatment of a human or non-human animal, more particularly for use as an anti-viral, anti-tumour or immunomodulatory agent;
a pharmaceutical composition comprising such a polynucleotide and a pharmaceutically acceptable carrier or diluent;
a method of treating a subject having a Type 1 interferon treatable disease, which method comprises administering to said patient an effective amount of such a polynucleotide;
use of such a polynucleotide in the manufacture of a medicament, e.g. a vector preparation, for use in therapy as an anti-viral, anti-tumour or immunomodulatory agent, more particularly for use in treating a Type 1 interferon treatable disease; and
a method of identifying a compound having immunomodulatory activity and/or anti-viral activity and/or anti-tumour activity comprising providing a cell capable of expressing HuIFRG 198 protein or a naturally occurring variant thereof, incubating said cell with a compound under test and monitoring for upregulation of HuIFRG 198 gene expression.

In a still further aspect, the invention provides a method of predicting responsiveness of a patient to treatment with a Type 1 interferon, e.g. IFN-α treatment (such as IFN-α treatment by the oromucosal route or a parenteral route, for example, intravenously, subcutaneously, or intramuscularly), which comprises determining the level of HuIFRG 198 protein or a naturally-occurring variant thereof, e.g. an allelic variant, or the corresponding mRNA, in a cell sample from said patient, e.g. a blood sample, wherein said sample is obtained from said patient following administration of a Type 1 interferon, e.g. IFN-α by an oromucosal route or intravenously, or is treated prior to said determining with a Type 1 interferon such as IFN-α in vitro. The invention also extends to kits for carrying out such testing.

### BRIEF DESCRIPTION OF THE SEQUENCES

SEQ. ID. No.1 is the amino acid sequence of human protein HuIFRG 198 and its encoding cDNA.

SEQ. ID. No.2 is the amino acid sequence alone of HuIFRG 198 protein.

### DETAILED DESCRIPTION OF THE INVENTION

As indicated above, human protein HuIFRG 198 and functional variants thereof are now envisaged as therapeutically useful agents, more particularly for use as an anti-viral, anti-tumour or immunomodulatory agent.

A variant of HuIFRG 198 protein for this purpose may be a naturally occurring variant, either an allelic variant or species variant, which has substantially the same functional activity as HuIFRG 198 protein and is also upregulated in response to administration of IFN-α. Alternatively, a variant of HuIFRG 198 protein for therapeutic use may comprise a sequence which varies from SEQ. ID. No. 2 but which is a non-natural mutant.

The term “functional variant” refers to a polypeptide which has the same essential character or basic function of HuIFRG 198 protein. The essential character of HuIFRG 198 protein may be deemed to be as an immunomodulatory peptide. A functional variant polypeptide may show additionally or alternatively anti-viral activity and/or anti-tumour activity.

Desired anti-viral activity may, for example, be tested or monitored as follows. A sequence encoding a variant to be tested is cloned into a retroviral vector such as a retroviral vector derived from the Moloney murine leukemia virus (MoMuLV) containing the viral packaging signal Ψ, and a drug-resistance marker. A pantropic packaging cell line containing the viral gag, and pol, genes is then co-transfected with the recombinant retroviral vector and a plasmid, pVSV-G, containing the vesicular stomatitis virus envelope glycoprotein in order to produce high-titre infectious replication incompetent virus (Burns et al., Proc. Natl. Acad. Sci. USA 84, 5232-5236). The infectious recombinant virus is then used to transfect interferon sensitive fibroblasts or lymphoblastoid cells and cell lines that stably express the variant protein are then selected and tested for resistance to virus infection in a standard interferon bio-assay (Tovey et al., Nature, 271, 622-625, 1978). Growth inhibition using a standard proliferation assay (Mosmann, T., J. Immunol. Methods, 65, 55-63, 1983) and expression of MHC class I and class II antigens using standard techniques may also be determined.

A desired functional variant of HuIFRG 198 may consist essentially of the sequence of SEQ. ID. No.2. A functional variant of SEQ. ID. No.2 may be a polypeptide which has a least 60% to 70% identity, preferably at least 80% or at least 90% and particularly preferably at least 95%, at least 97% or at least 99% identity with the amino acid sequence of SEQ. ID. No.2 over a region of at least 20, preferably at least 30, for instance at least 100 contiguous amino acids or over the full length of SEQ. ID. No.2. In a preferred aspect the invention relates to a functional variant of SEQ ID NO:2 which has greater than 98% identity, preferably at least 98.5%, at least 99% or at least 99.5% identity with the amino acid sequence of SEQ ID NO:2 over the full length of SEQ ID NO:2. Methods of measuring protein identity are well known in the art.

Amino acid substitutions may be made, for example from 1, 2 or 3 to 10, 20 or 30 substitutions. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Variant polypeptide sequences for therapeutic use in accordance with the invention may be shorter polypeptide sequences, for example, a peptide of at least 20 amino acids or up to 50, 60, 70, 80, 100, 150 or 200 amino acids in length is considered to fall within the scope of the invention provided it retains appropriate biological activity of HuIFRG 198 protein. In particular, but not exclusively, this aspect of the invention encompasses the situation when the variant is a fragment of a complete natural naturally-occurring protein sequence.

Also encompassed by the invention are modified forms of HuIFRG 198 protein and fragments thereof which can be used to raise anti-HuIFRG 198 protein antibodies. Such variants will comprise an epitope of the HuIFRG 198 protein.

Polypeptides of the invention may be chemically modified, e.g. post-translationally modified. For example, they may be glycosylated and/or comprise modified amino acid residues. They may also be modified by the addition of a sequence at the N-terminus and/or C-terminus, for example by provision of histidine residues or a T7 tag to assist their purification or by the addition of a signal sequence to promote insertion into the cell membrane. Such modified polypeptides fall within the scope of the term “polypeptide” of the invention.

A polypeptide of the invention may be labelled with a revealing label. The revealing label may be any suitable label which allows the polypeptide to be detected. Suitable labels include radioisotopes such as ¹²⁵I, ³⁵S or enzymes, antibodies, polynucleotides and linkers such as biotin. Labelled polypeptides of the invention may be used in assays. In such assays it may be preferred to provide the polypeptide attached to a solid support. The present invention also relates to such labelled and/or immobilised polypeptides packaged in the form of a kit in a container. The kit may optionally contain other suitable reagent(s), control(s) or instructions and the like.

The polypeptides of the invention may be made synthetically or by recombinant means. Such polypeptides of the invention may be modified to include non-naturally occurring amino acids, e.g. D amino acids. Variant polypeptides of the invention may have modifications to increase stability in vitro and/or in vivo. When the polypeptides are produced by synthetic means, such modifications may be introduced during production. The polypeptides may also be modified following either synthetic or recombinant production.

A number of side chain modifications are known in the protein modification art and may be present in polypeptides of the invention. Such modifications include, for example, modifications of amino acids by reductive alkylation by reaction with an aldehyde followed by reduction with NaBH₄, amidination with methylacetimidate or acylation with acetic anhydride.

Polypeptides of the invention will be in substantially isolated form. It will be understood that the polypeptides may be mixed with carriers or diluents which will not interfere with the intended purpose of the polypeptide and still be regarded as substantially isolated. A polypeptide of the invention may also be in substantially purified form, in which case it will generally comprise the polypeptide in a preparation in which more than 90%, for example more than 95%, 98% or 99%, by weight of polypeptide in the preparation is a polypeptide of the invention.

Polynucleotides

The invention also includes isolated nucleotide sequences that encode HuIFRG 198 protein or a variant thereof as well as isolated nucleotide sequences which are complementary thereto. The nucleotide sequence may be DNA or RNA, single or double stranded, including genomic DNA, synthetic DNA or cDNA. Preferably the nucleotide sequence is a DNA sequence and most preferably, a cDNA sequence.

As indicated above, such a polynucleotide will typically include a sequence comprising:
(a) the nucleic acid of SEQ. ID. No. 1 or the coding sequence thereof and/or a sequence complementary thereto;
(b) a sequence which hybridises, e.g. under stringent conditions, to a sequence complementary to a sequence as defined in (a);
(c) a sequence which is degenerate as a result of the genetic code to a sequence as defined in (a) or (b);
(d) a sequence having at least 60% identity to a sequence as defined in (a),(b) or (c).

In a preferred aspect, a polynucleotide of the invention encodes the HuIFRG 198 protein of SEQ ID NO:2 or a variant of said HuIFRG 198 protein having more than 98% identity with the sequence of SEQ ID NO:2 over the full length of SEQ ID NO:2. Such a polynucleotide may encode a functional variant of SEQ ID NO:2 having greater than 98% identity, preferably at least 98.5%, at least 99% or at least 99.5% identity with the amino acid sequence of SEQ ID NO:2 over the full length of SEQ ID NO:2.

Polynucleotides comprising an appropriate coding sequence can be isolated from human cells or synthesised according to methods well known in the art, as described by way of example in Sambrook et al. (1989) Molecular Cloning: A Laboratory Manual, 2^{nd} edition, Cold Spring Harbor Laboratory Press.

Polynucleotides of the invention may include within them synthetic or modified nucleotides. A number of different types of modification to polynucleotides are known in the art. These include methylphosphonate and phosphothioate backbones, addition of acridine or polylysine chains at the 3′ and/or 5′ ends of the molecule. Such modifications may be carried out in order to enhance the in vivo activity or lifespan of polynucleotides of the invention.

Typically a polynucleotide of the invention will include a sequence of nucleotides, which may preferably be a contiguous sequence of nucleotides, which is capable of hybridising under selective conditions to the coding sequence or the complement of the coding sequence of SEQ. ID. No.1. Such hybridisation will occur at a level significantly above background. Background hybridisation may occur, for example, because of other cDNAs present in a cDNA library. The signal level generated by the interaction between a polynucleotide of the invention and the coding sequence or complement of the coding sequence of SEQ. ID. No.1 will typically be at least 10 fold, preferably at least 100 fold, as intense as interactions between other polynucleotides and the coding sequence of SEQ. ID. No.1. The intensity of interaction may be measured, for example, by radiolabelling the probe, e.g. with ³²P. Selective hybridisation may typically be achieved using conditions of low stringency (0.3M sodium chloride and 0.03M sodium citrate at about 40° C.), medium stringency (for example, 0.3M sodium chloride and 0.03M sodium citrate at about 50° C.) or high stringency (for example, 0.03M sodium chloride and 0.03M sodium citrate at about 60° C.).

The coding sequence of SEQ ID No:1 may be modified by nucleotide substitutions, for example from 1, 2 or 3 to 10, 25, 50 or 100 substitutions. Degenerate substitutions may be made and/or substitutions may be made which would result in a conservative amino acid substitution when the modified sequence is translated, for example as shown in the table above. The coding sequence of SEQ. ID. NO: 1 may alternatively or additionally be modified by one or more insertions and/or deletions and/or by an extension at either or both ends.

A polynucleotide of the invention capable of selectively hybridising to a DNA sequence selected from SEQ. ID No.1, the coding sequence thereof and DNA sequences complementary thereto will be generally at least 70%, preferably at least 80 or 90% and more preferably at least 95% or 97%, homologous to the target sequence. This homology may typically be over a region of at least 20, preferably at least 30, for instance at least 40, 60 or 100 or more contiguous nucleotides.

Any combination of the above mentioned degrees of homology and minimum sized may be used to define polynucleotides of the invention, with the more stringent combinations (i.e. higher homology over longer lengths) being preferred. Thus for example a polynucleotide which is at least 80% homologous over 25, preferably over 30 nucleotides forms may be found suitable, as may be a polynucleotide which is at least 90% homologous over 40 nucleotides.

Homologues of polynucleotide or protein sequences as referred to herein may be determined in accordance with well-known means of homology calculation, e.g. protein homology may be calculated on the basis of amino acid identity (sometimes referred to as “hard homology”). For example the UWGCG Package provides the BESTFIT program which can be used to calculate homology, for example used on its default settings, (Devereux et al. (1984) Nucleic Acids Research 12, 387-395). The PILEUP and BLAST algorithms can be used to calculate homology or line up sequences or to identify equivalent or corresponding sequences, typically used on their default settings, for example as described in Altschul S. F. (1993) J. Mol. Evol. 36,290-300; Altschul, S. F. et al. (1990) J. Mol. Biol. 215, 403-10.

Software for performing BLAST analyses is publicly available through the National Center for Biotechnology Information (http://www.ncbi.nlm.nib.gov/). This algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89, 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Polynucleotides according to the invention have utility in production of the proteins according to the invention, which may take place in vitro, in vivo or ex vivo. In such a polynucleotide, the coding sequence for the desired protein of the invention will be operably-linked to a promoter sequence which is capable of directing expression of the desired protein in the chosen host cell. Such a polynucleotide will generally be in the form of an expression vector. Polynucleotides of the invention, e.g. in the form of an expression vector, which direct expression in vivo of a polypeptide of the invention having immunomodulatory activity and/or anti-viral activity and/or anti-tumour activity may also be used as a therapeutic agent

Expression vectors for such purposes may be constructed in accordance with conventional practices in the art of recombinant DNA technology. They may, for example, involve the use of plasmid DNA. They may be provided with an origin of replication. Such a vector may contain one or more selectable marker genes, for example an ampicillin resistance gene in the case of a bacterial plasmid. Other features of vectors of the invention may include appropriate initiators, enhancers and other elements, such as for example polyadenylation signals which may be desirable, and which are positioned in the correct orientation, in order to allow for protein expression. Other suitable non-plasmid vectors would be apparent to persons skilled in the art. By way of further example in this regard reference is made again to Sambrook et al., 1989 (supra). Such vectors additionally include, for example, viral vectors. Examples of suitable viral vectors include herpes simplex viral vectors, replication-defective retroviruses, including lentiviruses, adenoviruses, adeno-associated virus, HPV viruses (such as HPV-16 and HPV-18) and attenuated influenza virus vectors.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium and β-actin promoters. Viral promoters such as the SV40 large T antigen promoter or adenovirus promoters may also be used. Other examples of viral promoters which may be employed include the Moloney murine leukemia virus long terminal repeat (MMLV LTR), the rous sarcoma virus (RSV) LTR promoter, the human cytomegalovirus (CMV) IE promoter, and HPV promoters, particularly the HPV upstream regulatory region (URR). Other suitable promoters will be well-known to those skilled in the recombinant DNA art

An expression vector of the invention may further include sequences flanking the coding sequence for the desired polypeptide of the invention providing sequences homologous to eukaryotic genomic sequences, preferably mammalian genomic sequences, or viral genomic sequences. This will allow the introduction of such polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell.

The invention also includes cells in vitro, for example prokaryotic or eukaryotic cells, which have been modified to express the HuIFRG 198 protein or a variant thereof. Such cells include stable, e.g. eukaryotic, cell lines wherein a polynucleotide encoding HuIFRG 198 protein or a variant thereof is incorporated into the host genome. Host cells of the invention may be mammalian cells or insect cells, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa and COS cells. Preferably a cell line may be chosen which is not only stable, but also allows for mature glycosylation of a polypeptide. Expression may, for example, be achieved in transformed oocytes.

A polypeptide of the invention may be expressed in cells of a transgenic non-human animal, preferably a mouse. A transgenic non-human animal capable of expressing a polypeptide of the invention is included within the scope of the invention.

Polynucleotides according to the invention may also be inserted into vectors as described above in an antisense orientation in order to provide for the production of antisense sequences. Antisense RNA or other antisense polynucleotides may also be produced by synthetic means.

A polynucleotide, e.g. in the form of an expression vector, capable of expressing in vivo an antisense sequence to a coding sequence for the amino acid sequence defined by SEQ. ID. No.2, or a naturally-occurring variant thereof, for use in therapeutic treatment of a human or non-human animal is also envisaged as constituting an additional aspect of the invention. Such a polynucleotide will find use in treatment of diseases associated with upregulation of HuIFRG 198 protein.

Polynucleotides of the invention extend to sets of primers for nucleic acid amplification which target sequences within the cDNA for a polypeptide of the invention, e.g. pairs of primers for PCR amplification. The invention also provides probes suitable for targeting a sequence within a cDNA or RNA for a polypeptide of the invention which may be labelled with a revealing label, e.g. a radioactive label or a non-radioactive label such as an enzyme or biotin. Such probes may be attached to a solid support. Such a solid support may be a micro-array (also commonly referred to as nucleic acid, probe or DNA chip) carrying probes for further nucleic acids, e.g. mRNAs or amplification products thereof corresponding to other Type 1 interferon upregulated genes, e.g. such genes identified as upregulated in response to oromucosal or intravenous administration of IFN-α. Methods for constructing such micro-arrays are well-known (see, for example, EP-B 0476014 and 0619321 of Affymax Technologies N.V. and Nature Genetics Supplement January 1999 entitled “The Chipping Forecast”).

The nucleic acid sequence of such a primer or probe will preferably be at least 10, preferably at least 15 or at least 20, for example at least 25, at least 30 or at least 40 nucleotides in length. It may, however, be up to 40, 50, 60, 70, 100 or 150 nucleotides in length or even longer.

Another aspect of the invention is the use of probes or primers of the invention to identify mutations in HuIFRG 198 genes, for example single nucleotide polymorphisms (SNPs).

As indicated above, in a still further aspect the present invention provides a method of identifying a compound having immunomodulatory activity and/or antiviral activity and/or anti-tumour activity comprising providing a cell capable of expressing HuIFRG 198 protein or a naturally-occurring variant thereof, incubating said cell with a compound under test and monitoring for upregulation of HuIFRG 198 gene expression. Such monitoring may be by probing for mRNA encoding HuIFRG 198 protein or a naturally-occurring variant thereof. Alternatively antibodies or antibody fragments capable of specifically binding one or more of HuIFRG 198 and naturally-occurring variants thereof may be employed.

Antibodies

According to another aspect, the present invention also relates to antibodies (for example polyclonal or preferably monoclonal antibodies, chimeric antibodies, humanised antibodies and fragments thereof which retain antigen-binding capability) which have been obtained by conventional techniques and are specific for a polypeptide of the invention. Such antibodies could, for example, be useful in purification, isolation or screening methods involving immunoprecipitation and may be used as tools to further elucidate the function of HuIFRG 198 protein or a variant thereof. They may be therapeutic agents in their own right. Such antibodies may be raised against specific epitopes of proteins according to the invention. An antibody specifically binds to a protein when it binds with high affinity to the protein for which it is specific but does not bind or binds with only low affinity to other proteins. A variety of protocols for competitive binding or immunoradiometric assays to determine the specific binding capability of an antibody are well-known.

Pharmaceutical Compositions

A polypeptide of the invention is typically formulated for administration with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methyl cellulose, carboxymethylcellulose or polyvinyl pyrrolidone; desegregating agents, e.g. starch, alginic acid, alginates or sodium starch glycolate; effervescing mixtures; dyestuff; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tableting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methyl cellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusions may contain as carrier, for example, sterile water or preferably they may be in the form of sterile, aqueous, isotonic saline solutions.

A suitable dose of HuIFRG 198 protein or a functional analogue thereof for use in accordance with the invention may be determined according to various parameters, especially according to the substance used; the age, weight and condition of the patient to be treated; the route of administration; and the required regimen. Again, a physician will be able to determine the required route of administration and dosage for any particular patient A typical daily dose may be from about 0.1 to 50 mg per kg, preferably from about 0.1 mg/kg to 10 mg/kg of body weight, according to the activity of the specific inhibitor, the age, weight and condition of the subject to be treated, and the frequency and route of administration. Preferably, daily dosage levels may be from 5 mg to 2 g.

A polynucleotide of the invention suitable for therapeutic use will also typically be formulated for administration with a pharmaceutically acceptable carrier or diluent. Such a polynucleotide may be administered by any known technique whereby expression of the desired polypeptide can be attained in vivo. For example, the polynucleotide may be introduced by injection, preferably intradermally, subcutaneously or intramuscularly. Alternatively, the nucleic acid may be delivered directly across the skin using a particle-mediated delivery device. A polynucleotide of the invention suitable for therapeutic nucleic acid may alternatively be administered to the oromucosal surface for example by intranasal or oral administration.

A non-viral vector of the invention suitable for therapeutic use may, for example, be packaged into liposomes or into surfactant containing vector delivery particles. Uptake of nucleic acid constructs of the invention may be enhanced by several known transfection techniques, for example those including the use of transfection agents. Examples of these agents include cationic agents, for example calcium phosphate and DEAE dextran and lipofectants, for example lipophectam and transfectam. The dosage of the nucleic acid to be administered can be varied. Typically, the nucleic acid will be administered in the range of from 1 pg to 1 mg, preferably from 1 pg to 10 μg nucleic acid for particle-mediated gene delivery and from 10 μg to 1 mg for other routes.

Prediction of Type 1 Interferon Responsiveness

As also indicated above, in a still further aspect the present invention provides a method of predicting responsiveness of a patient to treatment with a Type 1 interferon, e.g. IFN-α treatment such as IFN-α treatment by an oromucosal route or intravenously, which comprises determining the level of HuIFRG 198 protein or a naturally-occurring variant thereof, or the corresponding mRNA, in a cell sample from said patient, wherein said sample is taken from said patient following administration of a Type 1 interferon or is treated prior to said determining with a Type 1 interferon in vitro.

Preferably, the Type 1 interferon for testing responsiveness will be the Type 1 interferon selected for treatment. It may be administered by the proposed treatment route and at the proposed treatment dose. Preferably, the subsequent sample analysed may be, for example, a blood sample or a sample of peripheral blood mononuclear cells (PBMCs) isolated from a blood sample.

More conveniently and preferably, a sample obtained from the patient comprising PBMCs isolated from blood may be treated in vitro with a Type 1 interferon, e.g. at a dosage range of about 1 to 10,000 IU/ml. Such treatment may be for a period of hours, e.g. about 7 to 8 hours. Preferred treatment conditions for such in vitro testing may be determined by testing PBMCs taken from normal donors with the same interferon and looking for upregulation of an appropriate expression product. Again, the Type 1 interferon employed will preferably be the Type 1 interferon proposed for treatment of the patient, e.g. recombinant IFN-α. PBMCs for such testing may be isolated in conventional manner from a blood sample using Ficoll-Hypaque density gradients. An example of a suitable protocol for such in vitro testing of Type 1 interferon responsiveness is provided in Example 3 below.

The sample, if appropriate after in vitro treatment with a Type 1 interferon, may be analysed for the level of HuIFRG 198 protein or a naturally-occurring variant thereof. This may be done using an antibody or antibodies capable of specifically binding one or more of HuIFRG 198 protein and naturally-occurring variants thereof, e.g. allelic variants thereof. Preferably, however, the sample will be analysed for mRNA encoding HuIFRG 198 protein or a naturally-occurring variant thereof. Such mRNA analysis may employ any of the techniques known for detection of mRNAs, e.g. Northern blot detection or mRNA differential display. A variety of known nucleic acid amplification protocols may be employed to amplify any mRNA of interest present in the sample, or a portion thereof, prior to detection. The mRNA of interest, or a corresponding amplified nucleic acid, may be probed for using a nucleic acid probe attached to a solid support. Such a solid support may be a micro-array as previously discussed above carrying probes to determine the level of further mRNAs or amplification products thereof corresponding to Type 1 interferon upregulated genes, e.g. such genes identified as upregulated in response to oromucosal or intravenous administration of IFN-α.

The following examples illustrate the invention:

### EXAMPLES

### Example 1

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon a (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi 1987, (Anal. Biochem. 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GG, AC, CA, GA, AG, CG or GC primers. Samples were then run on 7% denaturing polyacrylamide gels and exposed to authoradiography. Putative differentially expressed bands were cut out, reamplified according to the instructions of the supplier, and further used as probes to hybridize Northern blots of RNA extracted from the oropharyngeal cavity of IFN treated, IL-15 treated, and excipient treated animals.

Cloning and Sequencing

Re-amplified bands from the differential display screen were cloned in the Sfr 1 site of the pPCR-Script SK(+) plasmid (Stratagene) and cDNAs amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

Isolation of Human cDNA

Differentially expressed murine 3′ sequences identified from the differential display screen were compared with random human expressed sequence tags (EST) present in the dbEST database of GenBank® of the United States National Center for Biotechnology Information (NCBI). The sequences potentially related to the murine EST isolated from the differential display screen were combined in a contig and used to construct a human consensus sequence corresponding to a putative cDNA. One such cDNA was found to be 6045 nucleotides in length. This corresponded to a mouse gene whose expression was found to be enhanced approximately 3-fold in the lymphoid tissue of the oral cavity of mice following oromucosal administration of IFN-α.

In order to establish that this putative cDNA corresponded to an authentic human gene, primers derived from the 5′ and 3′ ends of the consensus sequence were used to synthesise cDNA from MRNA extracted from human peripheral blood leukocytes (PBL) by specific reverse transcription and PCR amplification. A unique cDNA fragment of the predicted size was obtained, cloned and sequenced (SEQ. ID. No.1). This human cDNA contains an open reading frame (ORF) of 5139 bp in length at positions 243 to 5381 encoding a protein of 1712 amino acids (SEQ. ID. No.2).

### Example 2

### Intravenous Administration of IFN-α

Male DBA/2 mice were injected intraperitoneally with 100,000 IU of recombinant murine IFN-α purchased from Life Technologies Inc. in 200 μl of PBS or treated with an equal volume of PBS alone. Eight hours later, the animals were sacrificed by cervical dislocation and the spleen was removed using conventional procedures. Total RNA was extracted by the method of Chomczynski and Sacchi (Anal. Biochem. (1987) 162, 156-159) and 10.0 μg of total RNA per sample was subjected to Northern blotting in the presence of glyoxal and hybridised with a cDNA probe for HuIFRG 198 mRNA as described by Dandoy-Dron et al.(J. Biol. Chem. (1998) 273, 7691-7697). The blots were first exposed to autoradiography and then quantified using a Phospholmager according to the manufacturer's instructions. Enhanced levels of mRNA for HuIFRG 198 protein (approximately 4 fold) were detected in samples of RNA extracted from spleens of IFN-α treated animals relative to animals treated with excipient alone.

### Example 3

### Testing Type 1 Interferon Responsiveness in vitro

Human Daudi or HeLa cells were treated in vitro with 10,000 IU of recombinant human IFN-α2 (Intron A from Schering-Plough) in PBS or with an equal volume of PBS alone. Eight hours later the cells were centrifuged (800×g for 10 minutes) and the cell pellet recovered. Total RNA was extracted from the cell pellet by the method of Chomczynski and Sacchi and 10.0 μg of total RNA per sample was subjected to Northern blotting in the presence of glyoxal and hybridised with a cDNA probe for HuIFRG 198 mRNA as previously described in Example 2 above. Enhanced levels of mRNA for HUIFRG 198 protein (approximately 3-fold) were detected in samples of RNA extracted from IFN-α treated Daudi or HeLa cells compared to samples treated with PBS alone.

The same procedure may be used to predict Type 1 interferon responsiveness using PBMCs taken from a patient proposed to be treated with a Type 1 interferon.

That which is claimed:

1. An isolated polypeptide comprising:
(i) the amino acid sequence of SEQ ID NO:2;
(ii) a variant thereof having substantially similar function selected from immunomodulatory activity and/or anti-viral activity and/or anti-tumour activity;
(iii) a fragment of (i) or (ii) which retains substantially similar function selected from immunomodulatory activity and/or anti-viral activity and/or anti-tumor activity; or
(iv) a variant or fragment of the polypeptide of (i) suitable for raising specific antibodies for said polypeptide and/or a naturally-occurring variant thereof.

2. An isolated polypeptide according to claim 1 comprising an amino acid sequence having more than 98% identity with the amino acid sequence of SEQ ID NO:2 over the full length of SEQ ID NO:2.

3. A polynucleotide encoding a polypeptide as claimed in claim 1.

4. A polynucleotide as claimed in claim 3 which is a cDNA.

5. A polynucleotide as claimed in claim 3, which polynucleotide comprises:
(a) the nucleic acid sequence of SEQ ID NO:1 or the coding sequence thereof and/or a sequence complementary thereto;
(b) a sequence which hybridises to a sequence as defined in (a);
(c) a sequence that is degenerate as a result of the genetic code to a sequence as defined in (a) or (b); or
(d) a sequence having at least 60% identity to a sequence as defined in (a), (b) or (c).

6. A composition of matter selected from the group consisting of:
(a) an expression vector comprising a polynucleotide sequence which is capable of expressing a polypeptide, where the polypeptide comprises:
(i) the amino acid sequence of SEQ ID NO:2;
(ii) a variant thereof having substantially similar function selected from immunomodulatory activity and/or anti-viral activity and/or anti-tumor activity; or
(iii) a fragment of (i) or (ii) which retains substantially similar function selected from immunomodulatory activity and/or anti-viral activity and/or anti-tumor activity,
(b) a host cell containing an expression vector of (a);
(c) an antibody specific for a polypeptide as defined in (a);
(d) a pharmaceutical composition comprising a polypeptide as defined in (a) and a pharmaceutically acceptable carrier or diluent;
(e) a pharmaceutical composition comprising a polynucleotide encoding a polypeptide as defined in (a) and a pharmaceutically acceptable carrier or diluent;
(f) a polynucleotide capable of expressing in vivo an antisense sequence to a coding sequence for the amino acid sequence defined by SEQ ID NO:2 or a naturally-occurring variant of said coding sequence for use in therapeutic treatment of a human or non-human animal;
(g) a set of primers for nucleic acid amplification which target sequences within a cDNA encoding a polypeptide as defined in (a);
(h) a nucleic acid probe derived from a polynucleotide encoding a polypeptide as defined in (a); and
(i) a non-human transgenic animal capable of expressing a polypeptide as defined in (a).

7. A composition of matter according to claim 6(h) wherein the probe is attached to a solid support.

8. A method selected from the group consisting of:
(a) a method of treating a patient having a Type 1 interferon treatable disease, which comprises administering to said patient an effective amount of a polypeptide, wherein the polypeptide comprises:
(i) the amino acid sequence of SEQ ID NO:2;
(ii) a variant thereof having substantially similar function selected from immunomodulatory activity and/or anti-viral activity and/or anti-tumor activity; or
(iii) a fragment of (i) or (ii) which retains substantially similar function selected from immunomodulatory activity and/or anti-viral activity and/or anti-tumor activity,
(b) a method of treating a patient having a Type 1 interferon treatable disease, which comprises administering to said patient an effective amount of a polynucleotide encoding a polypeptide as defined in (a);
(c) a method of producing a polypeptide as defined in (a), which method comprises culturing host cells containing an expression vector comprising a polynucleotide sequence which is capable of expressing said polypeptide under conditions suitable for obtaining expression of the polypeptide and isolating the said polypeptide;
(d) a method of identifying a compound having immunomodulatory activity and/or anti-viral activity and/or anti-tumour activity comprising providing a cell capable of expressing the polypeptide of SEQ ID NO:2 or a naturally-occurring variant thereof, incubating said cell with a compound under test and monitoring for upregulation of the gene encoding said polypeptide or variant; and
(e) a method of predicting responsiveness of a patient to treatment with a Type 1 interferon, which comprises determining the level of the protein defined by the amino acid sequence set forth in SEQ ID NO:2 or a naturally-occurring variant thereof, or the corresponding mRNA, in a cell sample from said patient, wherein said sample is obtained from said patient following administration of a Type 1 interferon or is treated prior to said determining with a Type 1 interferon in vitro.

9. A method as claimed in claim 8 wherein the interferon administered prior to obtaining said sample or used to treat said sample in vitro is the interferon proposed for treatment of said patient.

10. A method as claimed in claim 8 wherein a sample comprising peripheral blood mononuclear cells isolated from a blood sample of the patient is treated with a Type 1 interferon in vitro.

11. A method as claimed claim 8 wherein said determining comprises determining the level of mRNA encoding the protein defined by the sequence set forth in SEQ ID NO:2 or a naturally-occurring variant of said protein.


SEQUENCE LISTING (2 sequences)

SEQ ID NO: 1 - DNA, 6045 bp, Homo sapiens
  CDS (243)..(5381)
agcgggctgggtcctaggccaggtctggggtaacctggaacttccacctgggctctgcgctaggtctctgtttcactccctccccgcggggcgcgcagctcgcgggtctttggacaccaccggtcctgagtccgcggactgccattttcattaagaactgccacttagaggtaccaaaataaagggtatttgctacctttaatacttgccagttcaggttggaggcacaggcagcagcaagaatggaaagaaatgttcttacaacattttcacaggaaatgtcccagttaattttgaatgaaatgccaaaagctgaatattccagtttattcaatgattttgttgaatctgaattttttttgattgatggggattcattacttatcacatgtatctgtgagatatcatttaagcctgggcagaacctccatttcttctatctggttgaacgctatcttgtggatcttattagcaaaggaggacaattcaccatagttttcttcaaggatgccgagtatgcgtatttcaacttccctgaacttctttctttgagaactgctttaattcttcatcttcagaagaataccaccattgatgttcgaacaacattttcgagatgcttatcaaaagagtggggaagtttcttggaagagagttacccatatttcctgatagttgcagacgaaggcctgaacgatctacaaacacagcttttcaactttttaatcattcattcttgggcaaggaaggtcaacgttgtactttcctcagggcaagaatctgatgttctttgcctttatgcataccttcttccaagcatgtacagacaccagattttttcctggaagaataagcagaacattaaagatgcttatacaaccctgcttaaccagttggaaagatttaagctttcagcattagcacctctttttggaagtttaaaatggaataatattacggaagaggcacacaagactgtatctctgcttacacaagtctggccagaaggatctgacattcggcgtgtcttttgtgttacttcatgctcattatctttgagaatgtaccatcgctttttaggaaacagagagccctcctctggtcaggaaactgagatccaacaggtgaacagtaattgcttaaccctgcaggagatggaagatttgtgtaaactgcattgtctcactgtggtttttctactccatctgcctctttctcaaagagcttgtgctagagtcatcacttcccattgggctgaggacatgaagcctttattacaaatgaaaaagtggtgtgaatatttcatcttaagaaatatacatacttttgaattttggaatctgaatttaattcacctttctgacttaaatgatgagcttttgttgaagaatattgctttttactatgaaaatgaaaatgtaaaaggcctacatttgaatttgggagataccattatgaaagattatgaatatctctggaataccgtatcaaagttggtcagagactttgaggttggacagccatttcctctgagaacaacaaaagtttgttttcttggaaagaaaccatcaccaatcaaagacagctccaatgaaatggtgcccaatttgggttttattccaacgtcatcttttgtggttgataaatttgctggagatattttgaaagatttgccttttctaaagagtgatgatcctattgttacttcactggttaaacaaaaggaatttgatgaacttgtgcactggcattctcataaacccctgagtgatgattatgacaggtccaggtgtcagtttgatgaaaaatctagagaccctcgtgttcttagatctgtgcaaaagtatcatgttttccaacggttttatgggaattcattagaaacagtctcttcgaaaatcatcgtgactcaaactattaagtcaaagaaggattttagtgggcccaagagcaaaaaggcacacgagaccaaggctgaaataattgctagagagaataagaaaaggttatttgccagggaagaacaaaaggaagagcaaaagtggaatgctttgtcattttctattgaagagcaattgaaagaaaatttacactctggaataaagagcctggaagattttttgaaatcctgtaaaagtagctgtgtgaaacttcaggttgaaatggtggggttaactgcttgcttgaaagcctggaaagaacattgccgaagtgaagaaggtaaaaccacgaaagatttaagtatagctgttcaggtgatgaaaaggatccactccttgatggaaaaatactcagaacttttacaagaagatgatcggcaactcatagccagatgccttaagtatttaggatttgatgagttggcaagttctttacatccagcccaggatgcagaaaatgatgtaaaagtgaagaaaaggaataaatattcaattggcattgggccagctcggttccaactgcaatacatgggccattatttgatacgagatgagagaaaagacccagatcccagggtccaggattttattcccgacacatggcagcgagagctccttgatgttgtggataagaatgagtcagcagtgattgttgccccaacgtcctcaggcaaaacatatgcctcctactactgtatggagaaagtgctgaaggagagcgacgacggggtggtcgtgtacgttgcacccacaaaggcccttgttaatcaagtggcagcaactgttcagaatcgttttacgaaaaatctgccaagtggtgaagttctctgtggtgttttcaccagggagtatcgtcatgatgccttaaactgtcaggtacttattacagtgcctgcctgctttgaaattctgctgcttgctcctcatcgccaaaactgggtgaaaaagatcagatatgttatatttgatgaggttcattgtcttggtggagaaattggagcagaaatctgggaacatctccttgtcatgatccgatgtccctttttggctctttcagctaccataagtaatcctgaacatctcaccgagtggctacaatcggtaaaatggtactggaaacaagaagacaaaataattgaaaataataccgcttctaaaagacatgtgggtcgtcaggccggctttcccaaagactacttgcaagtaaaacaatcgtataaagttagacttgtgctctatggagagaggtataatgatctagagaagcatgtatgttcaataaaacatggtgacattcattttgatcattttcacccatgtgctgcactaacaacagatcatattgaaaggtatggattccctcctgatcttaccctttcacctcgagaaagcatccagctgtatgatgccatgtttcaaatttggaaaagttggcctcgggcccaggaactgtgcccagaaaacttcattcattttaacaataaattagtcattaaaaagatggatgctaggaaatatgaagagagtctaaaggcagaattaacaagttggattaaaaatggcaacgtagagcaggccagaatggtacttcagaatcttagtcctgaagcagatttgagtccagaaaacatgatcaccatgtttccacttctagttgaaaaactaaggaaaatggagaagttacctgcactattttttttattcaagttaggagctgtagaaaacgcagctgaaagtgtgagcactttcctaaagaaaaagcaggagacaaaaaggcctcccaaagctgataaagaagcccatgtcatggctaacaaacttcgaaaagttaaaaaatccatagagaaacaaaagatcatagatgaaaagagccagaaaaaaaccagaaatgtggatcaaagcctaatacatgaagctgaacatgataatctagtgaagtgtctagagaagaacctggaaatcccacaggactgcacatatgctgatcaaaaagcagtggacactgagactttgcagaaggtatttggtcgagtaaaatttgaaagaaaaggtgaagaattgaaagccttggcagaaaggggtattggatatcatcacagtgctatgagtttcaaagaaaaacaattagttgaaatcctctttagaaaaggatatcttagggtggtgacagctactggaacacttgctttaggtgtcaacatgccttgtaaatctgtggtttttgctcaaaactcagtctatctggatgcgttgaattatagacagatgtctggccgtgctggaagaagaggtcaagacctgatgggagatgtatatttctttgatattccattccccaaaataggaaaactcataaaatccaatgttcctgagctgagaggacacttccctctcagcataaccctggtcctgcgactcatgctgctggcttccaagggagatgacccagaggataccaaggcaaaggtgctatcagtgctaaagcattcattgctgtccttcaagcaacccagagtcatggacatgttaaaactttacttcctgttttctttgcagttcctggtgaaagagggctatttagatcaagaaggtaatcctatggggtttgctggacttgtatcacatttgcattatcatgaaccttctaatcttgtttttgtcagttttcttgtaaatggactcttccatgatctctgtcagccaaccaggaaaggctcaaaacatttttctcaagacgttatggaaaagctagtattagtattggcacatctctttggaagaagatattttccaccaaagttccaagatgcacacttcgagttttatcaatcaaaggtgttccttgatgatctccctgaggattttagtgatgctttagatgaatataacatgaaaattatggaggactttaccactttcctacgaattgtttccaaactggctgatatgaatcaggaatatcaactcccattgtcaaaaatcaaattcacaggtaaagaatgtgaagactctcaactcgtatctcatttgatgagctgcaaggaaggaagagtagcaatttcaccatttgtttgtctgtctgggaactttgatgatgatttgcttcgactagaaactccaaaccatgttactctaggcacaatcggtgtcaatcgctctcaggctccagtgctgttgtcacagaaatttgataaccgaggaaggaaaatgtcgcttaatgcctatgcactggatttctacaaacatggttccttgataggattagtccaggataacaggatgaatgaaggagatgcttattatttgttgaaggattttgcactcaccattaaatctatcagtgtttccttgcgtgagctatgtgaaaatgaagacgacaacgttgtcttagcctttgaacaactgagtacaactttttgggaaaagttaaacaaagtctaaaaacaaagtctatgcaaaccacttaaaaataattccatagtagtttttcaggtcacgtttttgattcttatgcttcttgccagaaatacattatgataaagtggaaatacattacgatgaagtggaaagagcaaacactttggaatcaaacagagttgcaatcaaacctgccatgttctgtcatgaatactcacaaattatttagtatacctgaatcttggtttctttttataactgagtaataatggttacatctcaggtagtttgaggattgactaaaaaaatgcgagaatgttgtatgtgactgaataacaatttttactctgcgaagccaaagtaaatataatattatcagtaactttatccccagtgtcagtatttataaaatgtttattaaggctagaaaaaatgaatacaatatcctgaaggtgaaatatattctcttcaattagcataaatatgatttacataagttagctatacagctattgagatagtactttctagtaaacttaaactactttttaaacatacattttgtgatgatttaacaaaaatatagagaatgatttgctttattgtaattgtatataagtgactggaaaagcacaaagaaataaagtgggttcgatctgttaaataaaaaaaaaaaaaaaaaaaaaaa

SEQ ID NO: 2 - protein, 1712 aa, Homo sapiens
MERNVLTTFSQEMSQLILNEMPKAEYSSLFNDFVESEFFLIDGDSLLITCICEISFKPGQNLHFFYLVERYLVDLISKGGQFTIVFFKDAEYAYFNFPELLSLRTALILHLQKNTTIDVRTTFSRCLSKEWGSFLEESYPYFLIVADEGLNDLQTQLFNFLIIHSWARKVNVVLSSGQESDVLCLYAYLLPSMYRHQIFSWKNKQNIKDAYTTLLNQLERFKLSALAPLFGSLKWNNITEEAHKTVSLLTQVWPEGSDIRRVFCVTSCSLSLRMYHRFLGNREPSSGQETEIQQVNSNCLTLQEMEDLCKLHCLTVVFLLHLPLSQRACARVITSHWAEDMKPLLQMKKWCEYFILRNIHTFEFWNLNLIHLSDLNDELLLKNIAFYYENENVKGLHLNLGDTIMKDYEYLWNTVSKLVRDFEVGQPFPLRTTKVCFLGKKPSPIKDSSNEMVPNLGFIPTSSFVVDKFAGDILKDLPFLKSDDPIVTSLVKQKEFDELVHWHSHKPLSDDYDRSRCQFDEKSRDPRVLRSVQKYHVFQRFYGNSLETVSSKIIVTQTIKSKKDFSGPKSKKAHETKAEIIARENKKRLFAREEQKEEQKWNALSFSIEEQLKENLHSGIKSLEDFLKSCKSSCVKLQVEMVGLTACLKAWKEHCRSEEGKTTKDLSIAVQVMKRIHSLMEKYSELLQEDDRQLIARCLKYLGFDELASSLHPAQDAENDVKVKKRNKYSIGIGPARFQLQYMGHYLIRDERKDPDPRVQDFIPDTWQRELLDVVDKNESAVIVAPTSSGKTYASYYCMEKVLKESDDGVVVYVAPTKALVNQVAATVQNRFTKNLPSGEVLCGVFTREYRHDALNCQVLITVPACFEILLLAPHRQNWVKKIRYVIFDEVHCLGGEIGAEIWEHLLVMIRCPFLALSATISNPEHLTEWLQSVKWYWKQEDKIIENNTASKRHVGRQAGFPKDYLQVKQSYKVRLVLYGERYNDLEKHVCSIKHGDIHFDHFHPCAALTTDHIERYGFPPDLTLSPRESIQLYDAMFQIWKSWPRAQELCPENFIHFNNKLVIKKMDARKYEESLKAELTSWIKNGNVEQARMVLQNLSPEADLSPENMITMFPLLVEKLRKMEKLPALFFLFKLGAVENAAESVSTFLKKKQETKRPPKADKEAHVMANKLRKVKKSIEKQKIIDEKSQKKTRNVDQSLIHEAEHDNLVKCLEKNLEIPQDCTYADQKAVDTETLQKVFGRVKFERKGEELKALAERGIGYHHSAMSFKEKQLVEILFRKGYLRVVTATGTLALGVNMPCKSVVFAQNSVYLDALNYRQMSGRAGRRGQDLMGDVYFFDIPFPKIGKLIKSNVPELRGHFPLSITLVLRLMLLASKGDDPEDTKAKVLSVLKHSLLSFKQPRVMDMLKLYFLFSLQFLVKEGYLDQEGNPMGFAGLVSHLHYHEPSNLVFVSFLVNGLFHDLCQPTRKGSKHFSQDVMEKLVLVLAHLFGRRYFPPKFQDAHFEFYQSKVFLDDLPEDFSDALDEYNMKIMEDFTTFLRIVSKLADMNQEYQLPLSKIKFTGKECEDSQLVSHLMSCKEGRVAISPFVCLSGNFDDDLLRLETPNHVTLGTIGVNRSQAPVLLSQKFDNRGRKMSLNAYALDFYKHGSLIGLVQDNRMNEGDAYYLLKDFALTIKSISVSLRELCENEDDNVVLAFEQLSTTFWEKLNKV